(19) United States
(12) Patent Application Publication
(10) Pub. No.: US 2006/0058814 A1
(43) Pub. Date: Mar. 16, 2006

(54) Circumcision clamp

(75) Inventor: Gray A. Gillis, Ann Arbor, MI (US)

Correspondence Address: Christopher J. Fildes;Fildes & Outland, P.C., Suite 2, 20916 Mack Avenue, Grosse Pointe Woods, MI, 48236, US

(73) Assignee: Tri-State Hospital Supply Corporation

(21) Appl. No.: 10/940,540

(22) Filed: Sep. 14, 2004

(51) Int. Cl. A61B 17/32

(52) U.S. Cl. 606/118

(57) ABSTRACT

A circumcision clamp includes an elongated base member having a first end including a tapered aperture and a second end having an actuating means. The circumcision clamp further includes an elongated arm member having first and second ends. The first end has a receiver. The second end has cooperating actuating means cooperable with the base member actuating means. The arm member is pivotable on the base member. The circumcision clamp also includes an elongated clamp member having first and second ends. The first end includes an increased diameter portion terminating in an outwardly flared annular edge portion having a convexly curved surface adapted for engagement with the tapered aperture of the base member. The second end includes an engagement portion that is engagable with the receiver. Actuating the actuating means pivots the arm member to draw the convexly curved surface of the flared annular edge portion into the tapered aperture.

### TECHNICAL FIELD

This invention relates to surgical devices, and more particularly to an instrument for performing circumcision operations.

### BACKGROUND OF THE INVENTION

It is known in the art relating to circumcision operations to provide a clamping device to clamp and securely hold the prepuce of the penis prior to, during, and after surgical removal thereof. These circumcision devices are members of a group of circumcision devices referred to as the “bloodless” type. They clamp and squeeze the prepuce so as to prevent blood flow to this tissue that is to be cut and thereby removed. Since blood flow to the prepuce is stopped, little to no bleeding occurs at the wound formed by cutting the prepuce.

One such known “bloodless” circumcision clamp includes as essential parts a flat base member with a port in one end and a rod-shaped member enlarged into the form of a bell at one end. The dimensions of the two members are related so that a major portion of the bell can pass easily through the port in the base member, but the lip of the bell is made large enough that it will not pass through the port. As a consequence, when the smaller end of the bell member is inserted into the port and the member is drawn through as far as it will go, the lip of the bell presses tightly around the circumference of the port. A separate lever arm member is tightened to the base member and the bell member to secure bell to the port. In tightening the bell member into the base member, however, the bell member may not be clamped evenly all around as the bell is drawn into the base at an angle due to the geometry of the clamping.

In performing a circumcision operation using such a clamping device, the prepuce is drawn over the bell-shaped end of the bell member. The bell member is then inserted into the port of the base member as far as it will go and drawn tight by suitable means to clamp the prepuce between the lip of the bell and the rim of the port.

These circumcision clamps also conventionally include a pin or cylindrical lugs disposed perpendicular to the axis of the bell member near an end of the bell member opposite the bell. The pin or lugs are sufficiently longer than the diameter of the clamp member to cause its/their ends to engage grooves disposed at an end of the lever arm member. It is cumbersome, however, for a user to align and engage the pin or lugs with the grooves when attempting to secure the clamp member. If the pin or lugs are not exactly aligned with the grooves, the clamp member cannot be secured to the arm member.

### SUMMARY OF THE INVENTION

The present invention provides an improved circumcision clamp that is easier to manipulate and secure during surgical use.

A circumcision clamp in accordance with the present invention includes an elongated base member having first and second ends. The first end includes a tapered aperture and the second end has an actuating means. The circumcision clamp further includes an elongated arm member having first and second ends. The arm member is pivotable on the base member between the first and second ends. The arm member first end has a receiver that includes a concave surface. The arm member second end has cooperating actuating means cooperable with the base member actuating means. The circumcision clamp also includes an elongated clamp member having first and second ends. The clamp member first end includes an increased diameter portion terminating in an outwardly flared annular edge portion adapted for engagement with the tapered aperture of the base member. The clamp member second end includes a spherical engagement portion that is engagable with the concave surface of the receiver. Actuating the actuating means pivots the arm member to draw the flared annular edge portion against the tapered aperture.

Optionally, the spherical engagement portion may be a round ball or may be a portion of a round ball. The clamp member first end may be a hollow, bell-shaped cup having an opening on one end. The clamp member first end may have a hole therein its side. In an alternative embodiment, the circumcision clamp may include a clamp member including a flared annular edge portion having a convexly curved surface. The tapered aperture of the base member may have an endless wall with a curvilinear cross-sectional shape. The curvilinear cross-sectional shape may be generally concave or may be a generally straight taper.

The actuating means may include a threaded rod and a thumb nut removably engaged with the threaded rod. The threaded rod may be mounted at the second end of the base member and the arm member may include a slot for receiving the threaded rod. The base member and the arm member may be generally the same length.

In a specific embodiment, a circumcision clamp in accordance with the present invention includes an elongated base member extending longitudinally and having opposed first and second ends. The base member includes a tapered aperture positioned at the first end and a threaded rod projecting from the second end. The circumcision clamp further includes an elongated arm member extending longitudinally and having opposed first and second ends. The arm member is pivotable on the base member between the first and second ends. The arm member first end is offset from the arm member second end and includes a bifurcation defining two prongs and a slot therebetween. The bifurcation includes a concave surface. The arm member further includes a slot positioned at the second end adapted for receiving the threaded rod. The circumcision clamp also includes a thumb nut removably engaged with the threaded rod. The circumcision clamp further includes a clamp member including an elongated rod having opposed first and second ends, an increased diameter portion, an outwardly flared annular edge portion, and a spherical engagement portion. The increased diameter portion and the flared annular edge portion are positioned at the first end of the clamp member and the spherical engagement portion is positioned at the second end. The flared annular edge portion is adapted for engagement with the tapered aperture of the base member. The elongated rod is receivable in the arm member first end slot. The spherical engagement portion is engagable with the concave surface of the bifurcation. Actuating the thumb nut and threaded rod assembly draws the flared annular edge portion against the tapered aperture.

Optionally, in this specific embodiment the spherical engagement portion may be a round ball or may be a portion of a round ball. The clamp member first end may be a hollow, bell-shaped cup having an opening on one end. The clamp member first end may have a hole therein its side. Alternatively, the circumcision clamp may include a clamp member including a flared annular edge portion having a convexly curved surface. The tapered aperture of the base member may have an endless wall with a curvilinear cross-sectional shape. The curvilinear cross-sectional shape may be generally concave. The arm member may include a transversely extending rounded projection and the base member may include a transversely extending curved groove adapted for receiving the rounded projection. The base member and the arm member may be generally the same length.

A method of clamping a prepuce of the penis includes the step of providing a circumcision clamp including an elongated base member having first and second ends. The first end includes a tapered aperture and the second end has an actuating means. The circumcision clamp further includes an elongated arm member having first and second ends. The arm member is pivotable on the base member between the first and second ends. The arm member first end has a receiver that includes a concave surface. The arm member second end has cooperating actuating means cooperable with the base member actuating means. The circumcision clamp also includes an elongated clamp member having first and second ends. The clamp member first end includes an increased diameter portion terminating in an outwardly flared annular edge portion adapted for engagement with the tapered aperture of the base member. The clamp member second end includes a spherical engagement portion that is engagable with the concave surface of the receiver. Actuating the actuating means pivots the arm member to draw the flared annular edge portion against the tapered aperture.

Optionally, the step of providing a circumcision clamp may include providing a circumcision clamp wherein the spherical engagement portion is a round ball. Alternatively, the step of providing a circumcision clamp may include providing a circumcision clamp wherein the spherical engagement portion is a portion of a round ball.

These and other features and advantages of the invention will be more fully understood from the following detailed description of the invention taken together with the accompanying drawings.

### BRIEF DESCRIPTION OF THE DRAWINGS

In the drawings:

FIG. 1 is a perspective view of a circumcision clamp in accordance with the present invention;

FIG. 2 is a side elevational view of the circumcision clamp of FIG. 1;

FIG. 3 is a cross-sectional view of the circumcision clamp of FIG. 1;

FIG. 4 is a side elevational view of a clamp member of the circumcision clamp of FIG. 1;

FIG. 5 is a side elevational view of an alternative clamp member of a circumcision clamp in accordance with the present invention;

FIG. 6 is a perspective view of an arm member of the circumcision clamp of FIG. 1;

FIG. 7 is a side elevational view of an alternative embodiment of a clamp member of a circumcision clamp in accordance with the present invention;

FIG. 8 is a fragmentary cross-sectional view of a circumcision clamp in accordance with the present invention having the clamp member of FIG. 7; and

FIG. 9 is a fragmentary cross-sectional view of an alternative embodiment of a base member of a circumcision clamp in accordance with the present invention.

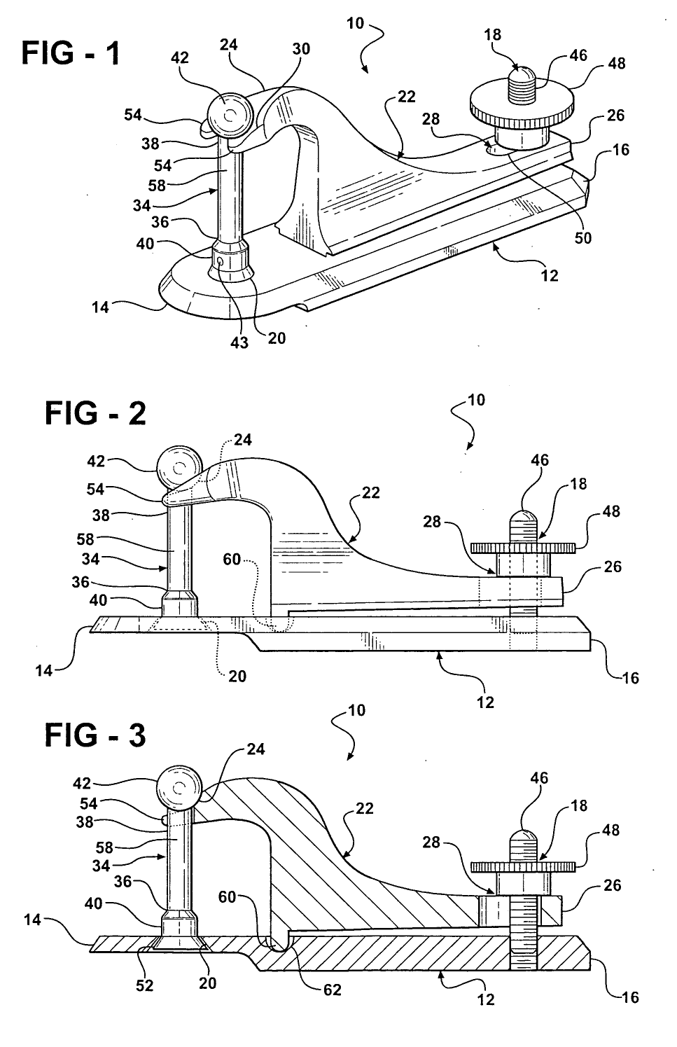

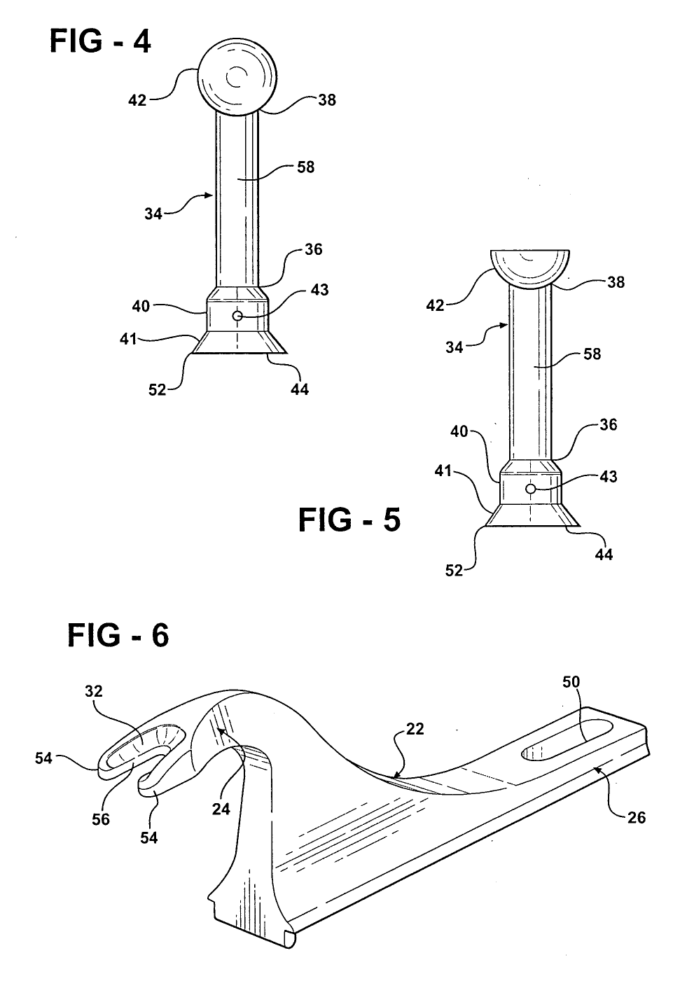

### DETAILED DESCRIPTION OF THE INVENTION

Referring now to the drawings in detail, numeral 10 generally indicates an improved circumcision clamp that is easier to manipulate and secure during surgical use.

The circumcision clamp 10 includes an elongated base member 12 having a first end 14 and a second end 16. The first end 14 includes a tapered aperture 20 and the second end 16 has an actuating means 18. The circumcision clamp 10 further includes an elongated arm member 22 having a first end 24 a second end 26. The arm member 22 is pivotable on the base member 12 between the first end 14 and second end 16. The arm member first end 24 has a receiver 30 that may include a concave surface 32. The arm member second end 26 has cooperating actuating means 28 cooperable with the base member actuating means 18. The circumcision clamp 10 also includes an elongated clamp member 34 having a first end 36 and a second end 38. The clamp member first end 36 includes an increased diameter portion 40 that terminates in an outwardly flared annular edge portion 41 having a convexly curved surface. The flared annular edge portion 41 is adapted for engagement with the tapered aperture 20 of the base member 12. The clamp member second end 38 includes an engagement portion 42 that is engagable with the receiver 30. Actuating the actuating means 18, 28 pivots the arm member 22 to draw the convexly curved surface of the flared annular edge portion 41 against the tapered aperture 20.

Optionally, the elongated clamp member second end 38 may include a spherical engagement portion 42 engagable with a concave surface 32 of the receiver 30. The spherical engagement portion 42 may be a round ball or may be a portion of a round ball. The spherical engagement portion 42 may, however, be any shape such that the portion has a spherical outer surface suitable to engage the concave surface 32 of the receiver 30. For example, the spherical engagement portion 42 may alternatively be a hollow hemisphere. The diameter of the increased diameter portion 40 is smaller than the diameter of the tapered aperture 20 such that the increased diameter portion 40 may pass through the tapered aperture 20. The clamp member first end 36 may also have a hole 43 therein its side.

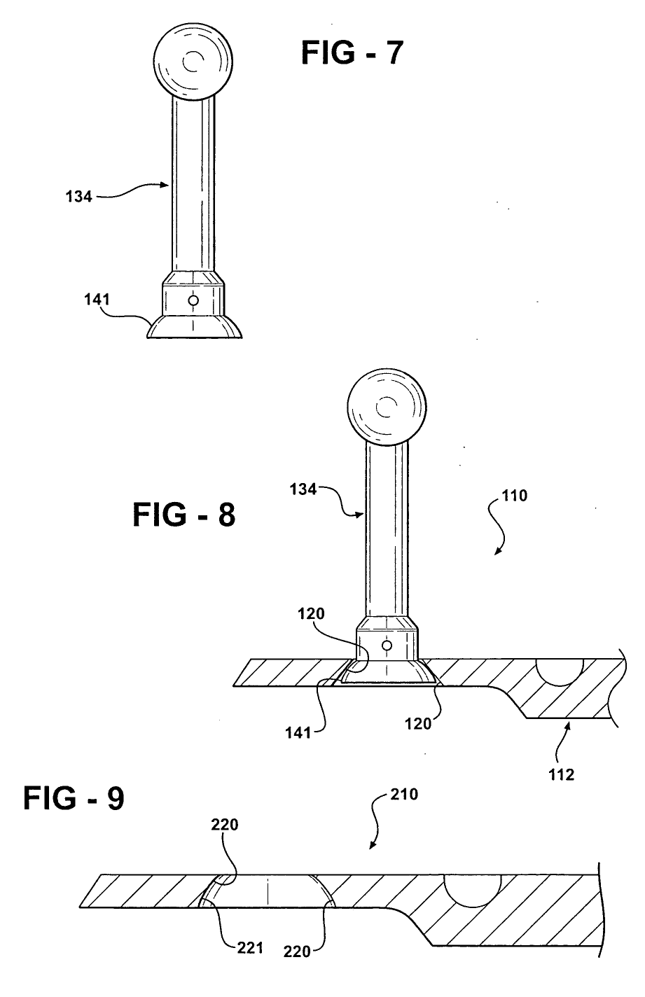

Further, the clamp member first end 36 may be a hollow, bell-shaped cup having an opening 44 on one end, or may be any other suitable cup-like shape. It is only necessary that the flared annular edge portion 41 have an annular edge 52 that is at least as large in diameter as the tapered aperture 20 such that the flared annular edge portion 41 is engagable with the tapered aperture. The flared annular edge portion 41 may have a taper that is cooperatively angled similar to the taper of the tapered aperture 20. In an alternative embodiment, as shown in FIGS. 7 and 8, a circumcision clamp 110 may include a clamp member 134 including a flared annular edge portion 141 having a convexly curved surface. The curved surface of the flared annular edge portion 141 provides a self aligning feature resulting in 360 degrees of contact between the flared annular edge portion 141 and the tapered aperture 120 of the base member 112 even when an axis of the clamp member 134 is not aligned relative to the tapered aperture 120. Further, the curved surface of the flared annular edge portion 141 insures that the force applied between the flared annular edge portion 141 and the tapered aperture 20 is even around a circumference of the flared annular edge portion and the tapered aperture. In yet another embodiment, as shown in FIG. 9, a circumcision clamp 210 may include a tapered aperture 220 of a base member 212 having an endless wall 221 with a curvilinear cross-sectional shape. The curvilinear cross-sectional shape may be generally concave.

The actuating means 18, 28 may include a threaded rod 46 and a thumb nut 48 removably engaged with the threaded rod 46. The threaded rod 46 may be mounted at the second end 16 of the base member 12 and the arm member 22 may include a slot 50 for receiving the threaded rod 46. Other suitable actuating means 18, 28 may be, but are not limited to, a threaded rod and wing nut assembly, a cam lever and toggle link assembly, and a cooperating serrated locking mechanism. The actuating means 18, 28 merely must be capable of causing the arm member 22 to pivot about the base member 12 and to draw and hold the flared annular edge portion 41 against the tapered aperture 20. Furthermore, the base member 12 and the arm member 22 may be generally the same length.

In a specific embodiment, the elongated base member 12 extends longitudinally and the first end 14 and second end 16 are in an opposed relationship. The tapered aperture 20 is positioned at the base member first end 14 and the threaded rod 46 projects from the base member second end 16. The elongated arm member 22 also extends longitudinally and the first end 24 and second end 26 are in an opposed relationship. The arm member 22 is pivotable on the base member 12 between the first and second ends. The arm member first end 24 is offset from the arm member second end 26 such that the arm member first end has a “goose-neck” appearance. The arm member first end 24 includes a receiver 30 that is a bifurcation defining two prongs 54 and a slot 56 therebetween. The bifurcation may include a concave surface 32. The arm member actuating means slot 50 is positioned at the second end 26 and is adapted for receiving the threaded rod 46. A thumb nut 48 is removably engaged with the threaded rod 46. The clamp member 34 includes an elongated rod 58 having a first end 36 and a second end 38 in an opposed relationship, an increased diameter portion 40, a flared annular edge portion 41, and an engagement portion 42. The increased diameter portion 40 and the flared annular edge portion 41 are positioned at the first end 36 of the clamp member 34 and the spherical engagement portion 42 is positioned at the second end 38. The flared annular edge portion 41 has a convexly curved surface and is adapted for engagement with the tapered aperture 20 of the base member 12. The elongated rod 58 is receivable in the arm member first end slot 56. The engagement portion 42 is engagable with the bifurcation. Actuating the thumb nut 48 and threaded rod 46 assembly draws the convexly curved surface of the flared annular edge portion 41 against the tapered aperture 20.

Optionally, in this specific embodiment the elongated clamp member second end 38 may include a spherical engagement portion 42 engagable with a concave surface 32 of the receiver 30. The spherical engagement portion 42 may be a round ball or a portion of a round ball. The spherical engagement portion 42 may, however, be any shape such that the portion has a spherical outer surface suitable to engage the concave surface 32 of the receiver 30. For example, the spherical engagement portion 42 may alternatively be a hollow hemisphere.

The clamp member first end 36 may be a hollow, bell-shaped cup having an opening 44 on one end, or may be any other suitable cup-like shape. It is only necessary that the flared annular edge portion 41 have an annular edge 52 that is at least as large in diameter as the tapered aperture 20 such that the flared annular edge portion is engagable with the tapered aperture. The flared annular edge portion 41 may have a taper that is cooperatively angled similar to the taper of the tapered aperture 20. For example, the tapered aperture 20 may have an endless wall with a straight tapered cross-sectional shape. In an alternative embodiment, as shown in FIGS. 7 and 8, a circumcision clamp 110 may include a clamp member 134 including a flared annular edge portion 141 having a convexly curved surface. In yet another embodiment, as shown in FIG. 9, a circumcision clamp 210 may include a tapered aperture 220 of a base member 212 having an endless wall 221 with a curvilinear cross-sectional shape. The curvilinear cross-sectional shape may be generally concave.

Further, the diameter of the increased diameter portion 40 is smaller than the diameter of the tapered aperture 20 such that the increased diameter portion 40 may pass through the tapered aperture 20. The clamp member first end 36 may also have a hole 43 therein its side.

The arm member 22 may include a transversely extending rounded projection 60 and the base member 12 may include a transversely extending curved groove 62 adapted for receiving the rounded projection 60. The rounded projection 60 may rest in the curved groove 62 and the curved groove may function as a fulcrum for the arm member 22. The base member 12 and the arm member 22 may also be generally the same length.

A method of clamping a prepuce of the penis includes the step of providing a circumcision clamp 10 including an elongated base member 12 having a first end 14 and a second end 16. The first end 14 includes a tapered aperture 20 and the second end 16 has an actuating means 18. The circumcision clamp 10 further includes an elongated arm member 22 having a first and 24 a second end 26. The arm member 22 is pivotable on the base member 12 between the first end 14 and second end 16. The arm member first end 24 has cooperating actuating means 28 cooperable with the base member actuating means 18. The arm member second end 26 has a receiver 30 that may include a concave surface 32. The circumcision clamp 10 also includes an elongated clamp member 34 having a first end 36 and a second end 38. The clamp member first end 36 includes an increased diameter portion 40 that terminates in an outwardly flared annular edge portion 41 having a convexly curved surface adapted for engagement with the tapered aperture 20 of the base member 12. The clamp member second end 38 includes an engagement portion 42 that is engagable with the receiver 30. Actuating the actuating means 18, 28 pivots the arm member 22 to draw the convexly curved surface of the flared annular edge portion 41 against the tapered aperture 20.

Optionally, the step of providing a circumcision clamp 10 may further include providing a circumcision clamp wherein the tapered aperture 20 of the base member has an endless wall with a generally concave curvilinear cross-sectional shape. The step of providing a circumcision clamp 10 may further include providing a circumcision clamp wherein the receiver 30 includes a concave surface 32 and the elongated clamp member second end 38 includes a spherical engagement portion 42 engagable with the concave surface of the receiver.

Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims.

What is claimed is:

1. A circumcision clamp comprising:
an elongated base member having first and second ends;
said first end including a tapered aperture;
said second end having an actuating means;
an elongated arm member having first and second ends; said arm member being pivotable on said base member between said first and second ends;
said first end having a receiver;
said second end having cooperating actuating means cooperable with said actuating means;
an elongated clamp member having first and second ends;
said first end including an increased diameter portion terminating in an outwardly flared annular edge portion having a convexly curved surface adapted for engagement with said tapered aperture;
said second end including an engagement portion being engagable with said receiver;
whereby actuating said actuating means pivots said arm member to draw said convexly curved surface of said flared annular edge portion against said tapered aperture.

2. The circumcision clamp of claim 1, wherein said tapered aperture of said base member has an endless wall with a generally concave curvilinear cross-sectional shape.

3. The circumcision clamp of claim 1, wherein said receiver includes a concave surface.

4. The circumcision clamp of claim 3, wherein said elongated clamp member second end includes a spherical engagement portion engagable with said concave surface of said receiver.

5. The circumcision clamp of claim 1, wherein said clamp member first end is a hollow, bell-shaped cup having an opening on one end.

6. The circumcision clamp of claim 1, wherein said clamp member first end has a hole therein its side.

7. The circumcision clamp of claim 1, wherein said actuating means comprises a threaded rod and a thumb nut, said thumb nut being removably engaged with said threaded rod.

8. The circumcision clamp of claim 7, wherein said threaded rod is mounted at said second end of said base member and said arm member includes a slot for receiving said threaded rod.

9. The circumcision clamp of claim 1, wherein said base member and said arm member are generally the same length.

10. A circumcision clamp comprising:
an elongated base member extending longitudinally and having opposed first and second ends;
said base member comprising a tapered aperture positioned at said first end and a threaded rod projecting from said second end;
an elongated arm member extending longitudinally and having opposed first and second ends;
said arm member being pivotable on said base member between said first and second ends;
said arm member first end being offset from said arm member second end and comprising a bifurcation defining two prongs and a slot therebetween;
said arm member comprising a slot positioned at said second end adapted for receiving said threaded rod;
a thumb nut removably engaged with said threaded rod; and
a clamp member comprising an elongated rod having opposed first and second ends, an increased diameter portion, an outwardly flared annular edge portion, and an engagement portion; said increased diameter portion and said flared annular edge portion positioned at said first end and said spherical engagement portion positioned at said second end;
said flared annular edge portion having a convexly curved surface adapted for engagement with said tapered aperture;
said elongated rod being receivable in said arm member first end slot;
said engagement portion being engagable with said bifurcation;
whereby actuating said thumb nut and threaded rod assembly draws said convexly curved surface of said flared annular edge portion against said tapered aperture.

11. The circumcision clamp of claim 10, wherein said tapered aperture of said base member has an endless wall with a generally curvilinear cross-sectional shape.

12. The circumcision clamp of claim 11, wherein said curvilinear cross-sectional shape is generally concave.

13. The circumcision clamp of claim 10, wherein said tapered aperture of said base member has an endless wall with a generally straight tapered cross-sectional shape.

14. The circumcision clamp of claim 10, wherein said bifurcation includes a concave surface.

15. The circumcision clamp of claim 14, wherein said clamp member includes a spherical engagement portion engagable with said concave surface of said bifurcation.

16. The circumcision clamp of claim 10, wherein said clamp member first end is a hollow, bell-shaped cup having an opening on one end.

17. The circumcision clamp of claim 10, wherein said clamp member first end has a hole therein its side.

18. The circumcision clamp of claim 10, wherein said arm member comprises a transversely extending rounded projection and said base member comprises a transversely extending curved groove adapted for receiving said rounded projection.

19. The circumcision clamp of claim 10, wherein said base member and said arm member are generally the same length.

20. A method of clamping a prepuce of the penis comprising the step of:
providing a circumcision clamp comprising:
an elongated base member having first and second ends;
said first end including a tapered aperture;
said second end having an actuating means;
an elongated arm member having first and second ends; said arm member being pivotable on said base member between said first and second ends;
said first end having a receiver;
said second end having cooperating actuating means cooperable with said actuating means;
an elongated clamp member having first and second ends;
said first end including an increased diameter portion terminating in an outwardly flared annular edge portion having a convexly curved surface adapted for engagement with said tapered aperture;
said second end including an engagement portion being engagable with said receiver;
whereby actuating said actuating means pivots said arm member to draw said convexly curved surface of said flared annular edge portion against said tapered aperture.

21. The method of claim 20, wherein the step of providing a circumcision clamp further comprises providing a circumcision clamp wherein said tapered aperture of said base member has an endless wall with a generally concave curvilinear cross-sectional shape.

22. The method of claim 20, wherein the step of providing a circumcision clamp further comprises providing a circumcision clamp wherein said receiver includes a concave surface and said elongated clamp member second end includes a spherical engagement portion engagable with said concave surface of said receiver.
